(19) United States
(12) Patent Application Publication
(10) Pub. No.: US 2017/0211438 A1
(43) Pub. Date: Jul. 27, 2017

(54) CPAP DEVICE

(71) Applicant: NIDEC COPAL ELECTRONICS CORPORATION, Tokyo (JP)

(72) Inventors: Takashi SUZUKI, Iruma-shi (JP); Yuki NAKADA, Iruma-shi (JP); Takatoshi INOGUCHI, Iruma-shi (JP); Yasuhiro TOBINAI, Iruma-shi (JP); Nobuo TAKAIWA, Iruma-shi (JP)

(73) Assignee: NIDEC COPAL ELECTRONICS CORPORATION, Tokyo (JP)

(21) Appl. No.: 15/328,217

(22) PCT Filed: Jul. 31, 2015
(86) PCT No.: PCT/JP2015/003871

§ 371 Date: Jan. 23, 2017

(30) Foreign Application Priority Data
Aug. 4, 2014 (JP) 2014-158639

(51) Int. Cl. F01N 1/08; A61M 16/08; A61M 16/06; A61M 16/00

(52) U.S. Cl. CPC F01N 1/08; A61M 16/0066; A61M 16/0875; A61M 16/0666; A61M 2205/42; A61M 2016/0027; A61M 2205/3365; A61M 2016/003

(57) ABSTRACT

There is provided a CPAP device in which an outflow sound of air is reduced while size reduction/weight reduction is maintained. The CPAP device includes a blower unit 10 which houses a turbofan 50 to draw in air and sends out the air and in which an air outflow opening to allow the air sent out from the turbofan 50 to flow out is formed, and a silencer 60 which is attached to one end of a hose 70 to feed the air flowing out from the air outflow opening to a mask 200 attached to a patient, is attachably and detachably attached to the air outflow opening of the blower unit 10 and reduces the outflow sound of the air flowing out from the air outflow opening, and the silencer 60 includes a regulating plate 69.

### TECHNICAL FIELD

The present invention is related to a CPAP (Continuous Positive Airway Pressure) device which is used for treatment of Sleep Apnea Syndrome.

### BACKGROUND ART

For treatment of Sleep Apnea Syndrome, there have been used CPAP devices which forcibly feed air into a respiratory tract by a fan while putting a mask or a nasal cannula on a face. As such a CPAP device, there has been generally adopted a configuration in which a main unit housing a fan is placed at a position away from a human body, and the main unit and the mask which is put on a face are connected by a hose therebetween and air is fed in through the hose. Masks and the like having various shapes have been developed and put onto the market, and a patient arbitrarily chooses and uses a mask which fits for its face shape and matches its preferences.

In a case of a CPAP device of such configuration, since there are a number of problems such as ones in which the hose is required to be cleaned regularly and the main unit has a size inconvenient for carrying, and since such device is inconvenient for a patient to handle it, contrary to that the treatment method is required to be used every day, such device often becomes one of treatment devices which are not used continuously.

In the Patent Literature 1, an endeavor in which it is intended to provide a CPAP device aiming for size reduction/weight reduction and being convenient for carrying is attempted.

### PRIOR ART LITERATURES

### Patent Literatures

Patent Literature 1: Japanese Laid-open Patent Publication No. 2013-150684

### ABSTRACT OF THE INVENTION

### Technical Problem

The CPAP device is a device which is used while a patient is sleeping and is required to be silent. For this reason, in the CPAP device, a fan is housed in a housing, and further, a structure to reduce an inflow sound of air is provided between an air inflow opening of the housing and the fan.

Here, in order to improve silence, it is preferable that, also for an outflow sound of the air sent out from the fan, a structure to reduce the outflow sound is provided. However, trying to reduce the outflow sound of the air may make the device large in size, and may be against the requirements of size reduction/weight reduction.

In view of the foregoing, it is an object of the present invention to provide a CPAP device in which the outflow sound of the air is reduced while size reduction/weight reduction is maintained.

### Solution to Problem

A CPAP device according to the present invention to obtain the above object includes:
a fan that draws in air and sends out the air; and
a silencer that reduces an outflow sound of the air sent out from the fan, and feeds the air whose outflow sound is reduced in a hose connected to a mask or a nasal cannula attached to a patient, wherein
the silencer includes a regulating plate which allows the air to pass therethrough so as to make a flow of the air after the passing closer to a regulated flow than that before the passing.

The air sent out from the fan disperses indirection of the flow and is not stable. For this reason, vortexes or pressure variations occur in an air flow path. These vortexes or pressure variations cause noise. When the regulating plate is provided in the air flow path, the flow is regulated when the air passes through the regulating plate, and dispersion of the flow and the pressure variations are reduced. By means of this, a vortex producing area, a pressure varying area are restricted and sounds to be generated are thus reduced.

Since the CPAP device according to the present invention includes the silencer to reduce the outflow sound of the air sent out from the fan and includes the regulating plate provided in the silencer, it is possible to reduce a volume of a sound absorbing member without increasing a thickness or a length of the sound absorbing member which directly absorbs the outflow sound much enough to obtain a sufficient sound absorbing effect just by the sound absorbing member. Thus, according to the CPAP device of the present invention, the outflow sound of the air is reduced while size reduction/weight reduction is maintained.

Here, in the CPAP device according to the present invention, it is preferable that the device further includes a pressure sensor that measures a pressure of the air after passing through the regulating plate and a control circuit that controls a rotation speed of the fan according to the pressure of the air measured by the pressure sensor.

A patient receives designation of a pressure range of air from a doctor, and thus, when the CPAP device is operated, it is required to feed the air at a pressure within the designated pressure range.

For this reason, the configuration in which the pressure sensor is provided in the CPAP device, and the rotation speed of the fan is controlled according to the pressure measured by the pressure sensor is adopted.

Here, in order to measure the pressure of the air by the pressure sensor, it is preferable to measure the pressure of the air after passing through the regulating plate. Since the air having passed through the regulating plate has small air pressure variations, and in addition, the pressure loss occurs in the regulating plate, it is possible to measure a pressure approaching a pressure of the air fed to a patient, by performing the measurement at a position after passing through the regulating plate which position is closer to the patient.

Further, in the CPAP device according to the present invention, it is preferable that the device includes a first air pressure measurement room that is to be a measurement target for the pressure of the air by the pressure sensor and a first communication path that connects the air flow path after passing through the regulating plate with the first air pressure measurement room.

When the first pressure measurement room which is connected to the air flow path by the first communication path therebetween is provided separately from the air flow path, the air inside the first pressure measurement room indicates the pressure of the air after passing through the regulating plate, and simultaneously, the pressure variations are further reduced, and thus, it becomes possible to perform the pressure measurement more precisely. In addition, by providing the first pressure measurement room, the flow of the air in the air flow path is not disturbed compared with a configuration in which the pressure of the air on the air flow path is directly measured, and thus, it is possible to realize a smoother flow of the air.

Further, in the CPAP device according to the present invention, it is also a preferable mode that the deice includes a flow sensor that measures a flow rate of the air flowing while passing through the regulating plate based on a differential pressure of the air before and after passing through the regulating plate, wherein the control circuit controls the rotation speed of the fan according to both the pressure of the air measured by the pressure sensor and the flow rate of the air measured by the flow sensor.

When the flow rate of the air is measured with the flow sensor provided, a patient may grasp a current condition of the patient such as whether the patient is at the timing of inspiration or at the timing of expiration. Thus, the flow sensor is provided in addition to the pressure sensor and the rotation speed of the fan is controlled according to the measurement results thereof, and thereby, it is possible to perform fine controlling to meet a condition of the patient every time.

The regulation effect by the regulating plate is compensation for the pressure loss, and the pressure loss is necessarily produced in the regulating plate. Thus, the flow sensor to measure the flow rate by measuring the differential pressure of the air before and after passing through the regulating plate is provided, and thereby, the measurement of the flow rate which highly precisely corresponds to the flow rate of the air fed to the patient is performed.

When the flow sensor is provided, it is preferable that the device includes a second air pressure measurement room for measuring the differential pressure of the air by the flow sensor and a second communicating path that connects the air flow path before passing through the regulating plate with the second air pressure measurement room, wherein the flow sensor measures the flow rate of the air flowing while passing through the regulating plate based on a differential pressure between the air inside the second air pressure measurement room and the air after passing through the regulating plate.

Similarly to the case of the above-described first pressure measurement room, the pressure of the air inside the second pressure measurement room stably reflects the pressure of the air before passing through the regulating plate. Accordingly, the second pressure measurement room is provided, and thus, the differential pressure of the air is stably measured. In addition, similarly to the case in which the first pressure measurement room is provided, the second pressure measurement room is provided, and thereby, it is prevented that the flow of the air is disturbed.

Further, in the CPAP device according to the present invention, it is preferable that the device includes a housing which houses the fan and in which an air outflow opening to allow the air sent out from the fan to flow out is formed, wherein the silencer is a silencer which is provided separately form the housing, is attached to one end of the hose and is attachably and detachably attached to the air outflow opening.

In the CPAP device of this mode, the silencer having the sound absorbing structure to reduce the outflow sound of the air is configured independently from the housing, and made to be attachable and detachable with respect to the housing. This silencer usually remains being attached to the one end of the hose. By means of this, since the housing is reduced in size and the silencer is attached to the hose, the silencer is housed integrally with the hose in a case for carrying and the like.

As described, according to the CPAP device of this mode, the device hardly becomes an obstacle for carrying and the like, and the outflow sound of the air is effectively reduced.

In addition, the hose is required to be cleaned regularly. Thus, in the case in which the silencer is configured to be separate from the housing, it is preferable that the silencer is also made to have a washable structure.

When the silencer is made to have a washable structure, the silencer may be cleaned together with the hose, and the silencer is also cleaned regularly.

In addition, in the case in which the pressure sensor is provided in the CPAP device according to the present invention, it is preferable that the device includes a housing which houses the fan and the pressure sensor and in which an air outflow opening to allow the air sent out from the fan to flow out is formed, wherein the silencer is a silencer which is provided separately from the housing, is attached to one end of the hose and is attachably and detachably attached to the air outflow opening, and the silencer includes a first air pressure transmitting path which is coupled to extend between a side of the silencer and a side of the housing by the attachment of the silencer to the air outflow opening, and transmits the pressure of the air after passing through the regulating plate to the pressure sensor.

The pressure sensor is required to be maintained in an atmospheric pressure ambiance and to guide the air of the pressure to be measured only to a specified section. Accordingly, when it is intended to provide the pressure sensor in the silencer configured to be separate from the housing, the silencer will become large in size. In addition, even if the pressure sensor is arranged in the silencer, a pressure measurement signal is required to be transmitted to a side of the housing. In contrast, since the CPAP device of this mode is provided with the above-described first air transmitting path and the pressure sensor is provided in the housing, electric components are gathered in the housing, and thus, the layout and the maintenance become easy.

In addition, in the case in which the flow sensor is provided in addition to the pressure sensor in the CPAP device according to the present invention, it is preferable that the deice includes a housing which houses the fan, the pressure sensor and the flow sensor, and in which an air outflow opening to allow the air sent out from the fan to flow out is formed, wherein, the silencer is a silencer which is provided separately from the housing, is attached to one end of the hose and is attachably and detachably attached to the air outflow opening, and the silencer includes a first air pressure transmitting path which is coupled to extend between the silencer and the housing by the attachment of the silencer to the air outflow opening, and transmits the pressure of the air after passing through the regulating plate to the pressure sensor and the flow sensor and a second air pressure transmitting path which is coupled to extend between a side of the silencer and the housing by the attachment of the silencer to the air outflow opening, and transmits a pressure of the air before passing through the regulated plate to the flow sensor.

The first air pressure transmitting path and the second air pressure transmitting path are provided and the pressure sensor and the flow sensor are arranged in the housing, and thereby, the layout and the maintenance become easy.

According to the above present invention, a CPAP device in which the outflow sound of the air is reduced while size reduction/weight reduction is maintained is realized.

### BRIEF DESCRIPTION OF THE DRAWINGS

FIG. 1 is a view of a whole configuration of a CPAP device as an embodiment according to the present invention.

FIG. 2 is an explaining view illustrating a usage state of the CPAP device illustrated in FIG. 1.

FIG. 3 is a perspective view of a silencer in a state of being attached to a blower unit.

FIG. 4 is a perspective view of the blower unit and the silencer in a state of being separated from each other while being arranged in their attaching postures.

FIG. 5 is a perspective view illustrating the blower unit and the silencer in a state of being separated from each other while being arranged in their attaching postures.

FIG. 6 is a side view of the blower unit and the silencer.

FIG. 7 is a control block diagram of the CPAP device according to the present embodiment.

FIG. 8 is an exploded perspective view of the blower unit with a bottom case opened illustrated while being upside down.

FIG. 9 is an exploded perspective view illustrating a configuration inside a first room of a housing of the blower unit.

FIG. 10 is an exploded perspective view illustrating the bottom case included in the housing of the blower unit and members to be housed in or attached to the bottom case.

FIG. 11 is a plan view illustrating an inner face of the bottom case in a state in which a second sound absorbing member, a drawing opening cover and others are assembled.

FIG. 12 is an exploded perspective view illustrating a configuration inside a second room of the housing of the blower unit.

FIG. 13 illustrates a side view of the blower unit when viewed from a side of an air outflow opening (Part (A)), and a cross sectional view along Arrows B-B illustrated in Part (A) of FIG. 13 (Part (B)).

FIG. 14 illustrates a side view of a discharging side silencer when viewed from a side of an air feeding opening to feed air in a hose (Part (A)), and a cross sectional view along Arrows C-C illustrated in Part (A) of FIG. 14 (Part (B)).

FIG. 15 illustrates a side view of the blower unit in a state of being attached with the silencer and the silencer when viewed from a side of the air feeding opening of the silencer (Part (A)), a cross sectional view along Arrows D-D illustrated in Part (A) of FIG. 15 (Part (B)) and a cross sectional view along Arrows E-E illustrated in Part (B) of FIG. 15 (Part (C)).

FIG. 16 is a cross sectional view of a portion of a regulating plate of the silencer.

FIG. 17 is a cross sectional view of a portion of the regulating plate of the silencer.

FIG. 18 is a cross sectional view of a portion of an end in a radius direction of a portion of the regulating plate of the silencer.

FIG. 19 is a cross sectional view of a portion of an end in the radius direction of a portion of the regulating plate in the silencer.

FIG. 20 is a view illustrating sound absorbing performance when a length of a flow path, a cross sectional shape and a thickness of the sound absorbing member are changed in various kinds by the inventors of the present invention.

FIG. 21 is a view illustrating sound absorbing performance ratio and a flow rate loss ratio with respect to a cross sectional shape coefficient t.

FIG. 22 is a view illustrating modified examples of a stretching way of wire to reduce a deformation of a second sound absorbing member.

FIG. 23 is a view illustrating modified examples of a first sound absorbing member.

FIG. 24 is a view illustrating a modified example of a drawing side silencer.

FIG. 25 is a perspective view illustrating a modified example of the discharging side silencer.

### BEST MODE FOR CARRYING OUT THE INVENTION

In the following, an embodiment of implementation of the present invention will be explained.

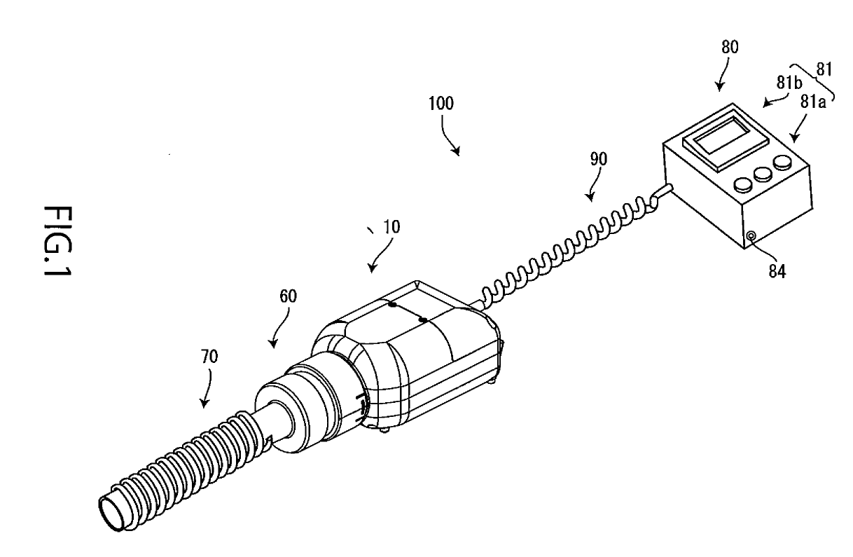

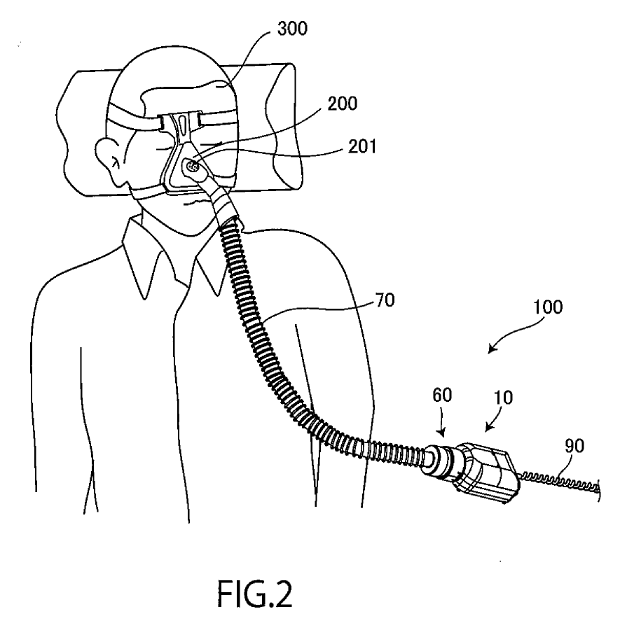

FIG. 1 is a view of a whole configuration of a CPAP device as one embodiment according to the present invention, and FIG. 2 is an explaining view illustrating a usage state of the CPAP device illustrated in FIG. 1. However, in FIG. 2, illustrations of the control unit 80 illustrated in FIG. 1 is omitted.

This CPAP device 100 includes a blower unit 10, a silencer 60, a hose 70, the control unit 80 and a cable 90. As illustrate in FIG. 2, the CPAP device 100 is used in a state in which the blower unit 10 and a mask 200 are connected by the hose 70 with the silencer 60, the mask 200 is put on a face of a patient 300, and the blower unit 10 is placed on bedclothes of the patient or on a side of the patient. The hose 70 is, for example, one having a length of about 50 centimeters.

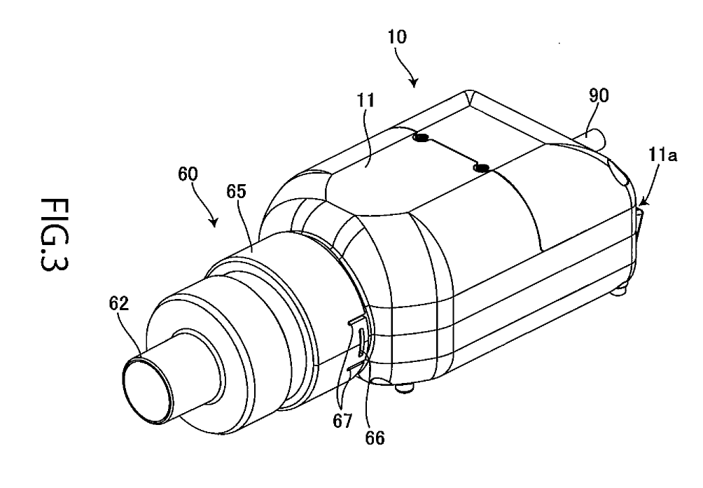

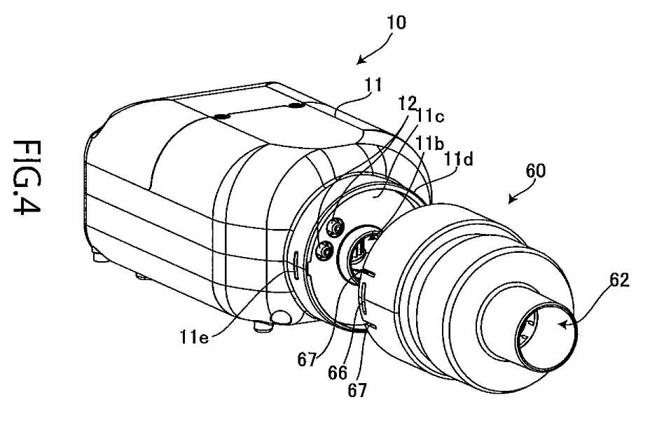

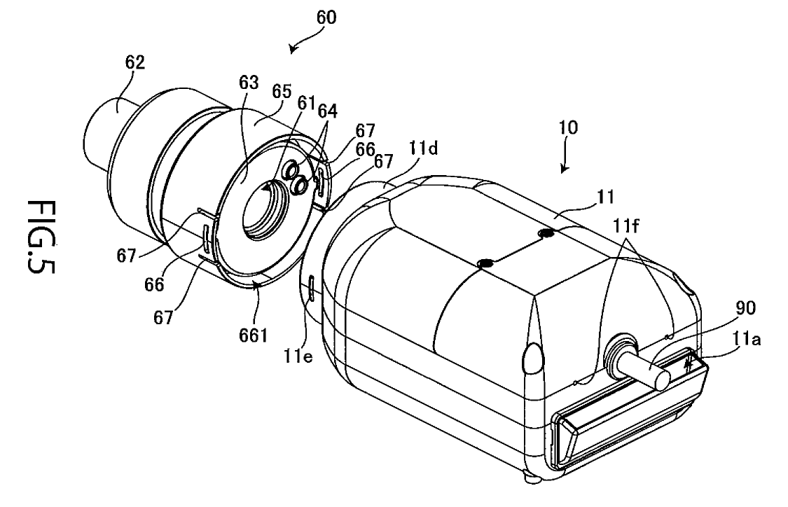

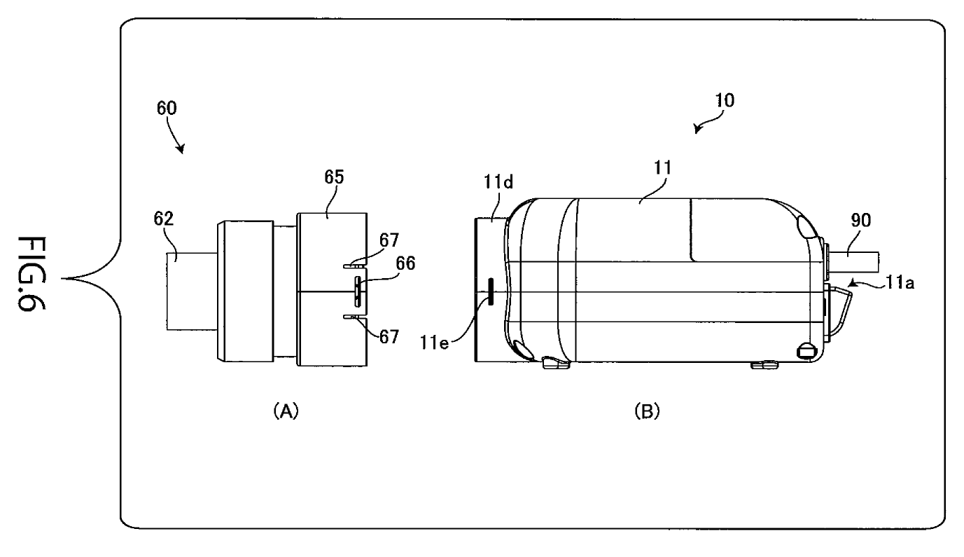

FIGS. 3 to 5 are perspective views of the blower unit and the silencer, and FIG. 6 is a side view of the blower unit and the silencer. Here, FIG. 3 is a perspective view of the silencer in a state of being attached to the blower unit, and FIGS. 4 and 5 are perspective views of the blower unit and the silencer in a state of being separated from each other while being arranged in their attaching postures. FIG. 6 is a side view of the state illustrated in FIGS. 4 and 5.

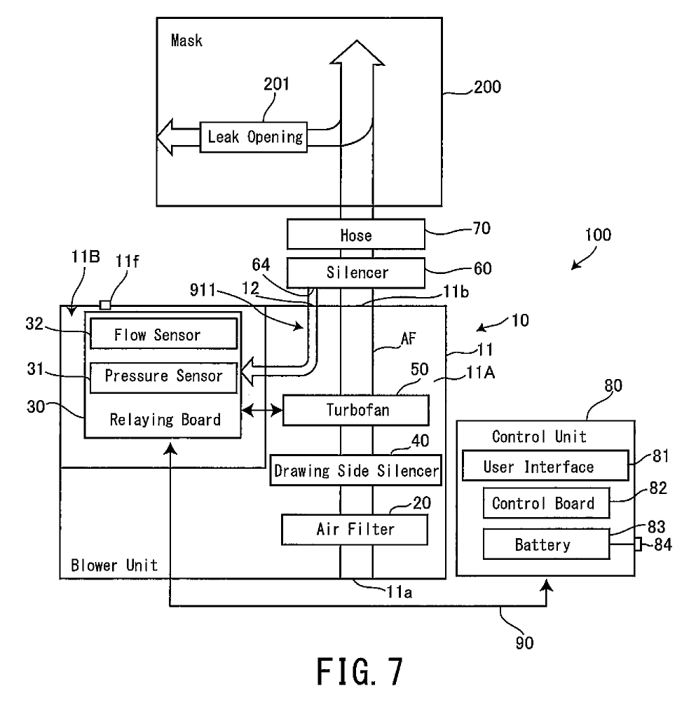

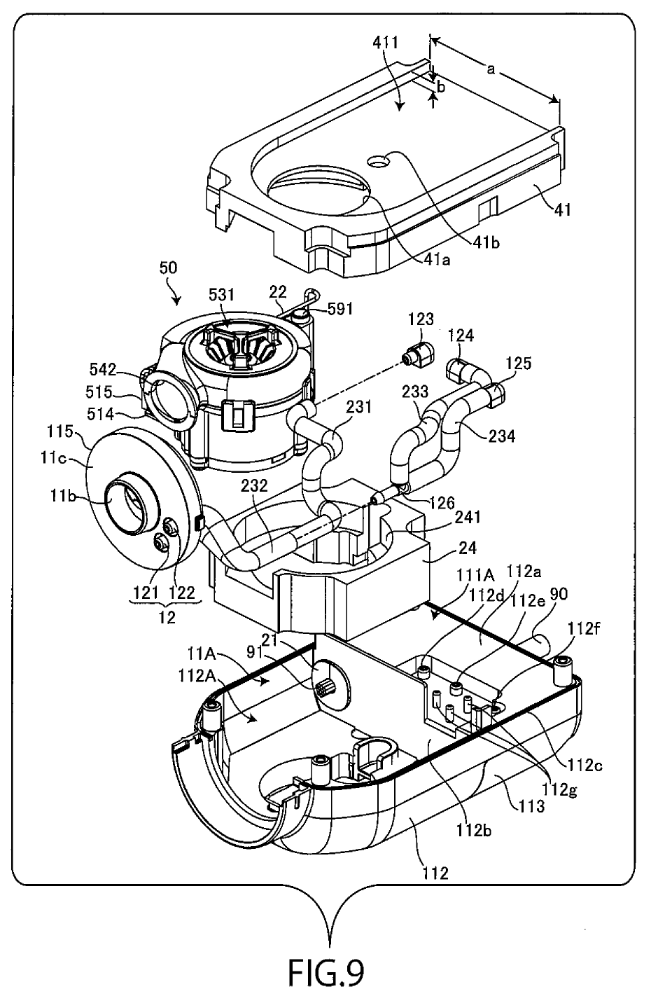

A turbofan 50 which will be described later (see FIGS. 7 and 9) is housed in a housing 11 of the blower unit 10. And in the housing 11, an air inflow opening 11a which allows air sent to the turbofan 50 to flow in an inside of the housing 11 and an air outflow opening 11b which projects in a cylindrical shape and allows the air sent out from the turbofan 50 to flow out are formed.

The silencer 60 is attachably and detachably attached to the air outflow opening 11b of the housing 11 of the blower unit 10 and plays a role of reducing an outflow noise of the air flowing out from the blower unit 10 and passing through the silencer 60. In the silencer 60, an air receiving opening 61 which is formed to be a circular opening to receive the air outflow opening 11b projecting in the cylindrical shape and receives the air flowing out from the air outflow opening 11b is formed. In addition, in the silencer 60, an air feeding opening 62 which projects in a cylindrical shape and feeds the air which has passed through the silencer 60 to the hose 70 is formed. The hose 70 is attached to the air feeding opening 62. When the CPAP device 100 is usually dismantled to be stored or carried, the silencer 60 is removed from the blower unit 10 while the silencer 60 is kept being attached to the hose 70.

When the silencer 60 is attached to the blower unit 10, the air outflow opening 11b of the blower unit 10 and the air receiving opening 61 of the silencer 60 are coupled with each other. As details will be explained later, two air pressure transmitting paths extending between the blower unit 10 and the silencer 60 are formed in the CPAP device 100. In the blower unit 10, two connectors 12 which show from an attaching surface 11c to the silencer 60 of the housing 11 are provided, and are arranged on end sections on the side of the blower unit 10 of those air pressure transmitting paths are provided. These two connectors 12 are connectors to couple portions on the side of blower unit 10 and portions on the side of silencer 60 of the two air pressure transmitting paths. In addition, correspondingly, two connectors 64 each having a cylindrical shape are provided on an attaching surface 63 to the blower unit 10 of the silencer 60. These two connectors 64 are provided on end sections on the side of the silencer 60 of the two air pressure transmitting paths. When the silencer 60 is attached to the blower unit 10, the air outflow opening 11b of the blower unit 10 and the air receiving opening 61 of the silencer 60 are connected with each other, and in addition, each of the two connectors 12 of the blower unit 10 and each of the two connectors 64 of the silencer 60 is coupled with each other, and thus, the two air pressure transmitting paths extending between the silencer 60 and the blower unit 10 are formed.

The attaching surface 11c of the blower unit 10 is surrounded by a coupling cylinder 11d having a cylindrical shape. In addition, the attaching surface 63 of the silencer 60 is also surrounded by a coupling cylinder 65 having a cylindrical shape. However, the coupling cylinder 65 of the silencer 60 has a size to allow the coupling cylinder 11d of the blower unit 10 to fit in an inside thereof, and a groove 661 which has a circular shape and which the coupling cylinder 11d of the blower unit 10 enters is provided between the attaching surface 63 and the coupling cylinder 65 of the silencer 60.

Locking projections 11e are respectively formed on both sides of an outer surface of the coupling cylinder 11d of the blower unit 10. On the other hand, locking openings 66 which the locking projections 11e enter are formed in the coupling cylinder 65 of the silencer 60. Nicks 67 are respectively formed on both sides of the respective locking openings 66, and portions of the locking openings 66 are formed in respective cantilever shapes by these nicks 67, thereby allowing appropriate bending.

When the silencer 60 is pressed against the blower unit 10 while the postures illustrated in FIGS. 4 and 5 are held, the coupling cylinder 11d of the blower unit 10 enters an inside of the coupling cylinder 65 of the silencer, and the locking projections 11e of the coupling cylinder 11d fit in the locking openings 66 of the coupling cylinder 65, and by means of this, the silencer 60 is attached to the blower unit 10.

When the silencer 60 is to be removed from the blower unit 10, the silencer 60 is pulled rather strongly while the blower unit 10 is held, and then the silencer 60 is removed from the blower unit 10.

FIG. 7 is a control block diagram of the CPAP device according to the present embodiment.

An air flow path AF which flows from the blower unit 10 via the silencer 60 and the hose 70 and further through the mask 200, and main constitutional elements of the CPAP device 100 are illustrated in here.

The blower unit 10 includes, on the air flow path AF, an air filter 20 to remove dust in air having flowed in from the air inflow opening 11a of the housing 11, a drawing side silencer 40 to reduce an inflow sound of the air and the turbofan 50 to send out the air, and since the turbofan 50 includes a rotor section including a blade and the like which rotor section is rotatably supported by an air dynamic pressure bearing, the turbofan 50 may rotate in a high-speed manner and size reduction/weight reduction are achieved. Incidentally, the silencer 60 which has been explained with reference to FIGS. 3 to 6 is different from the drawing side silencer 40, and the silencer 60 is a silencer on a discharging side to reduce outflow sounds of the air flowing out from the air outflow opening 11b of the housing 11, and is provided separately from the blower unit 10 and is attachably and detachably provided with respect to the blower unit 10.

The air sent out from the turbofan 50 flows out from the air outflow opening 11b of the housing 11, and is fed in the mask 200 via the silencer 60 on the discharging side and the hose 70. The air fed in the mask 200 is fed in a respiratory tract of a patient with respiration motions of the patient, and is discharged outside through a leak opening 201 (see also FIG. 2 together) by the respiration motions of the patient.

Here, the housing 11 of the blower unit 10 is partitioned into a first room 11A in which the above-described air filter 20, drawing side silencer 40 and turbofan 50 are arranged to form the air flow path AF, and a second room 11b in which a relaying board 30 which will be explained in the following is arranged. In addition, an opening 11f (see also FIG. 5 together) for keeping an inside of a second room 11B at atmospheric pressure is formed in the housing 11. Regarding the first room 11A, a pressure inside the first room 11A is varied by the operation of the turbofan 50. In contrast, since the second room 11B is kept be airtight with respect to the first room 11A and the opening 11f is formed, the second room 11B is always kept at atmospheric pressure regardless of the operation of the turbofan 50.

A pressure sensor 31 and a flow sensor 32 are provided on the relaying board 30 arranged in the second room 11B. As described above, in the blower unit 10 and silencer 60 on the discharging side, the air pressure transmitting paths 911 extending between them are provided. The air pressure transmitting paths 911 have an intermediate point which are connected by the coupling of the connector 12 on the side of the blower unit 10 and the connector 64 on the side of the silencer 60 on the discharging side. Air pressure of an inside of the silencer 60 on the discharging side is transmitted via the air pressure transmitting paths 911 to the pressure sensor 31 and the flow sensor 32, and pressures and flow rates of a portion thereof are measured. Measurement results thereof are transmitted to the control unit 80 via the cable 90. A user interface 81, a control board 82 and a battery 83 are housed in the control unit 80. In addition, an AC adaptor connecting terminal 84 (see also FIG. 1 together) is provided in the control unit 80.

As illustrated in FIG. 1, the user interface 81 includes plural operation buttons 81a and a display screen 81b. A patient operates the operation buttons 81a while checking with the display screen 81b, and sets a selection between a fixed mode and an automatic mode, a pressure range of air to be sent out from the turbofan 50 which pressure range is designated by a doctor, on-off timing of the turbofan 50 and the like. Here, the fixed mode is a mode in which a pressure of air to be sent out from the turbofan 50 is fixed to a designated pressure, and the automatic mode is a mode in which a breathing state of a patient is detected from changes of the pressure and the flow rates by the pressure sensor 31 and the flow sensor 32, the pressure is changed within the designated range according to the breathing state of the patient.

Information set by the user interface 81 is inputted to the control board 82. In addition, the air pressure and the air flow rate measured by the pressure sensor 31 and the flow sensor 32 are also inputted to the control board 82. In the control board 82, number of revolutions per unit time of the turbofan 50 is calculated based on those pieces of information. Then, a fan driving signal for causing the turbofan 50 to rotate at the calculated number of revolutions is generated and transmitted to the turbofan 50 via the cable 90 and the relaying board 30 in the blower unit 10. The turbofan 50 rotates at the number of revolutions according to the fan driving signal transmitted thereto.

In addition, the battery 83 housed in the control unit 80 is a battery having a capacity enough to allow the CPAP device 100 to operate for eight hours of sleeping duration of one time. The battery is provided, and thus, the device may be used even under a circumstance in which a commercial power source may not be obtained. The battery is charged from an AC adapter (not illustrated in the drawings) which is to be connected to the AC adapter connecting terminal 84.

In the following, detailed configurations of the blower unit and the silencer on the discharging side will be explained.

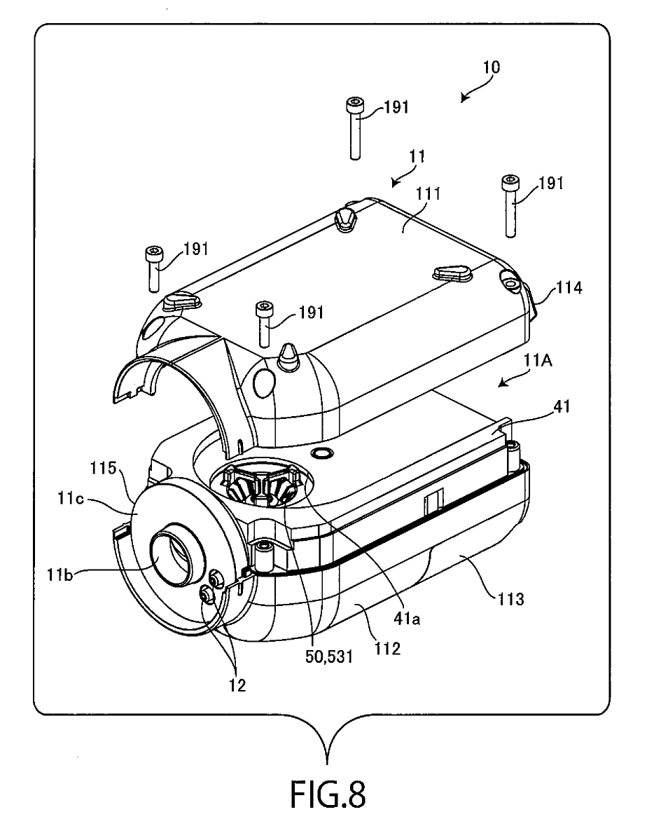

FIG. 8 is an exploded perspective view of the blower unit with a bottom case opened illustrated while being upside down.

The housing 11 of the blower unit 10 is composed of a bottom case 111, a main body case 112, a lid 113, a drawing opening cover 114 and a discharging opening cover 115. When the bottom case 111 is opened, the first room 11A (see also FIG. 7 together) in which the turbofan 50 and the like are housed appears inside that. In this FIG. 8, an air intake opening 531 of the turbofan 50 seen from an opening 41a provided in a ceiling side sound absorbing member 41 included in the drawing side silencer 40 (see FIG. 7) in the first room 11A is illustrated. Details will be described later. As illustrated in the drawings, the bottom case 111 is screwed to the main body case 112 with four screws 191. The coupling cylinder 11d having the cylindrical shape (see FIG. 6) on the side of the blower unit 10 is divided into two portions of the bottom case 111 and the main boy case 112, and the bottom case 111 is screwed to the main body case 112 so that the coupling cylinder 11d is formed to have the cylindrical shape. In addition, a surface on a side of the silencer 60 of the discharging opening cover 115 becomes the attaching surface 11c (see also FIG. 4 together) to the silencer 60.

The lid 113 included in the housing 11 is also screwed to the main body case 112. When the lid 113 is opened, the second room 11B (see FIG. 7) in an inside of which the relaying board 30 is housed appears. The second room 11B will be described later.

FIG. 9 is an exploded perspective view illustrating a configuration inside a first room of the housing of the blower unit. In this FIG. 9, illustrations of the bottom case 111 (see FIG. 8) are omitted. Similarly to FIG. 8, this FIG. 9 is also illustrated in an upside down manner.

The first room 11A is formed inside the main body case 112. Here, the second room 11B (see FIG. 7) does not appear in this FIG. 9, and the whole area illustrated in here represents the first room 11A. The second room 11B is a room which is surrounded by a bottom wall 112a of the main body case 112 which bottom wall 112a forms a floor of the second room 11B, a standing wall 112b and the lid 113, and which appears when the lid 113 is opened.

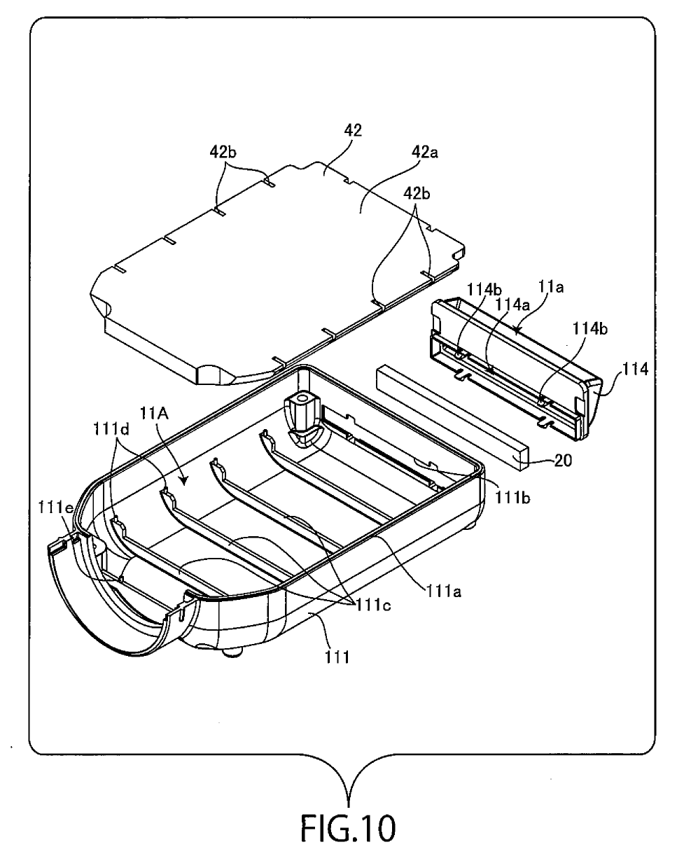

The first room 11A is divided into a first section 111A in which the drawing side silencer 40 (see FIG. 7) is mainly arranged and a second section 112A in which the turbofan 50 is arranged. The second room 11B vertically overlaps the first section 111A of the first room 11A. The second section 112A of the first room 11A does not overlap the second room 11B, and has a large volume to house the turbofan 50. As described, the second room 11B is overlapped with the first section 111A of the first room 11A in which first section 111A the drawing side silencer 40 is housed, and thus, a long air flow path required for absorbing sounds is secured between the air inflow opening 11a (see, for example, FIG. 5) and the turbofan 50. In addition, the second section 112A in which the large volume is secured without overlapped with the second room 11B is formed, and the turbofan 50 is housed in there. By means of these arrangements, size reduction of the blower unit 10 is achieved. The first room 11A and the second room 11B are connected with each other by wires 91 going through an opening (not illustrated in the drawings) provided in the standing wall 112b. In here, only a portion of the wires 91 which portion goes through the standing wall 112b is illustrated. The wires 91 are surrounded by a grommet 21 made of silicone rubber, and a leak of air from a circumference of the wires 91 is prevented. In addition, on an end surface of the main body case 112 which end surface contacts the bottom case 111, a groove 112c extending on almost all of the end surface except for a place at which the discharging opening cover 115 is arranged. In addition, a groove 111a (see FIG. 10) extending similarly is formed also on an end surface of the bottom case 111 which end surface contacts the main body case 112. Around cross section string 22 made of silicone rubber is arranged such that the round cross section string 22 fits in each of these grooves 112c and 111a. In addition, the discharging opening cover 115 is adhered to the main body case 112 and the bottom case 111. By means of this, it is prevented that air is drawn in from a portion other than the air inflow opening 11b (see FIG. 5) or air is blown out from a portion other than the air outflow opening 11b (see FIG. 4).

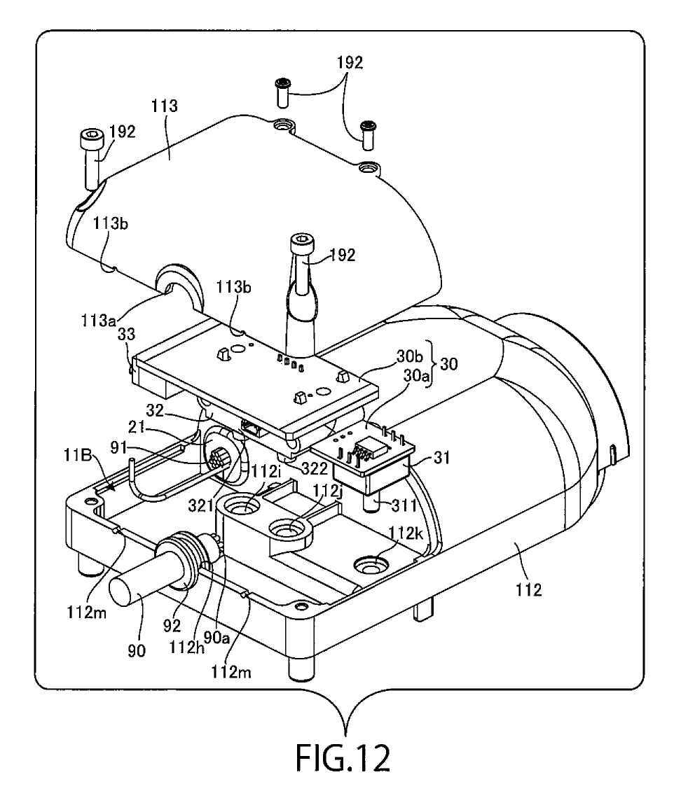

In addition, three bosses 112d, 112e and 112f are formed in the main body case 112. Openings 112i, 112j and 112k (see FIG. 12) to connect the first room 11A with the second room 11B are formed in respective centers of these three bosses 112d, 112e and 112f. Connectors 123, 124 and 125 which are respectively connected to one ends of silicone tubes 231, 233 and 234 are respectively connected to these bosses 112d, 112e and 112f. These silicone tubes 231, 233 and 234 and one more silicone tube 232 are members which form a portion on a side of the blower unit 10 of the air pressure transmitting path 911 (see FIG. 7) extending between the blower unit 10 and the discharging side silencer 60. The one end of the silicone tube 231 is connected to the connector 123 and the other end is connected to one connector 121 of the two connectors 12 coupling to the discharging side silencer 60. In addition, one end of the silicone tube 232 is connected to a connector 126 of branching type, and the other end is connected to the other connector 122 of the two connector 12. One ends of the remaining silicone tubes 233 and 234 are connected to the connectors 124 and 125, and each of the other ends is connected to the connector 126 of branching type. In other words, the two air pressure transmitting paths extend to the silencer 60 via the two connectors 12, and the silicone tube 231 which forms one of them is connected to the second room 11B via the connector 123. In addition, the other air pressure transmitting path is connected, via the silicone tube 232, while bifurcated by the connector 126, and further through the two silicone tubes 233 and 234, to the second room 11B via the respective connectors 124 and 125.

In addition, in the main body case 112, plural bosses 112g are further provided near the three bosses 112d, 112e and 112f to which the connectors 123, 124 and 125 are connected. These bosses 112g are for restricting passing routes of the silicone tubes 233 and 234.

A cover 24 composed of sponge to surround the turbofan 50 is arranged in the second section 112A, and the turbofan 50 is housed inside an opening 241 formed in the cover 24. The cover 24 plays a role of preventing vibrations as the turbofan 50 rotates from conducting to the housing 11. In addition, the cover 24 also plays a role of absorbing sounds. The turbofan 50 is arranged such that the turbofan 50 is surrounded by the cover 24, and an air discharging opening 542 thereof is connected to the air outflow opening 11b formed in the discharging opening cover 115 included in the housing 11. A circuit board 514 is provided in the turbofan 50, and a connector which is not illustrated in the drawings and is provided in a tip on a side of the first room 11A of the wires 91 extending from the second room 11B to the inside of the first room 11A is connected to a connector 515 provided on the circuit board 514.

In addition, the drawing side silencer 40 (see FIG. 7) is arranged in the first section 111A. A first sound absorbing member 41 of the sound absorbing members included in the drawing side silencer 40 is illustrated in this FIG. 9. In the first sound absorbing member 41, an air flow path 411 having a tabular shape of a width a and a height b is formed on a lower surface thereof (a surface oriented upward in FIG. 9). The first sound absorbing member 41 spreads up to a position overlapping the turbofan 50 housed in the second section 112A. And, two openings 41a and 41b are formed at positions overlapping the turbofan 50 in the first sound absorbing member 41. The opening 41a is an opening for connecting the air flow path 411 to the air intake opening 531 of the turbofan 50. In addition, the opening 41b is an opening for avoiding an interference of the turbofan 50 to the projection 591. The air flow path 411 having the tabular shape of the width a by the length b and being provided in the first sound absorbing member 41 will be studied in detail later.

FIG. 10 is an exploded perspective view illustrating the bottom case included in the housing of the blower unit and members to be housed in or attached to the bottom case.

The bottom case 111 is a component which forms the first room 11A together with the main body case 112. A second sound absorbing member 42 included in the drawing side silencer 40 (see FIG. 7) is arranged inside the bottom case 111. A surface 42a of the second sound absorbing member 42 which surface 42a is oriented to a side of the first sound absorbing member 41 (see FIG. 9) is formed to be flat. Accordingly, the air flow path 411 of the drawing side silencer 40 in which the first sound absorbing member 41 and the second sound absorbing member 42 are combined has a cross section of the width a by the length b formed in the first sound absorbing member 41.

In addition, an air intake opening 111b is formed in the bottom case 111. The drawing opening cover 114 in which the air inflow opening 11a is formed is attached to the air intake opening 111b such that the air filter 20 (see also FIG. 7 together) is sandwiched therebetween.

Plural ribs 111c for reinforcement are formed in an inside of the bottom case 111. Correspondingly, grooves (not illustrated in the drawings) for avoiding the ribs 111c are formed on a surface (a surface facing downward in FIG. 10) of the second sound absorbing member 42 which surface is on a side facing an inner wall surface of the bottom case 111. In addition, a projection 111d projecting toward an inside of the first room 11A is provided in each of both end sections in a length direction of each of the ribs 111c. Correspondingly, a slit 42b for allowing the projection 111d provided in each of the both end sections of each of the ribs 111c is formed at each of the both end sections of each of the grooves in the second sound absorbing member 42. In addition, a projection 111e is provided at a position on a downstream side of the flow of air in the bottom case 111. Further, projections 114b are provided on an upper edge of the opening 114a of the drawing opening cover 114 which opening 114a connects to the air intake opening 111b of the bottom case 111.

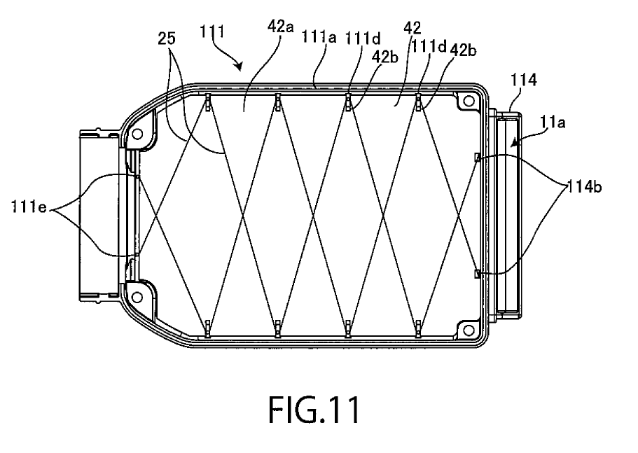

FIG. 11 is a plan view illustrating an inner face of the bottom case in a state in which the second sound absorbing member 42, the drawing opening cover 114 and others are assembled.

In here, a wire 25 such as a piano wire and the like is stretched by using the projections 111d of the bottom case 111 which projections 111d protrude from the slits 42b provided in the second sound absorbing member 42 and the other projections 111e and 114b (see also FIG. 10 together). The wire 25 is stretched around so as to go along the surface 42a of the second sound absorbing member 42 which surface 42a faces the first sound absorbing member 41 (see FIG. 9) and forms the air flow path 411 (see FIG. 9). The wire 25 is for preventing deformation of the second sound absorbing member 42. When air flows in the air flow path 411 which is formed between the sound absorbing member 41 and the second sound absorbing member 42 which are included in the drawing side silencer 40, an air pressure inside the air flow path 411 decreases, and a force in a direction to narrow the air flow path 411 is applied to the first sound absorbing member 41 and the second sound absorbing member 42. Thus, in the present embodiment, the wire 25 is stretched, and thereby, deformation of the second sound absorbing member 42 is prevented. For the first sound absorbing member 41, in the present embodiment, a sound absorbing material having rather hard quality and being not easily deformed is used even though the sound absorbing performance is decreased a little. In the present embodiment, by means of this, it is prevented that the air flow path 411 is squeezed, and an expected air flow path 411 is maintained.

FIG. 12 is an exploded perspective view illustrating a configuration inside the second room of the housing of the blower unit. In here, illustrations of constitutional elements inside the first room 11A (see FIG. 9) and the bottom case 111 (see FIG. 8) of the housing 11 are omitted.

As described above, when the lid 113 of the housing 11 is opened, the second room 11B surrounded by the lid 113 and the main body case 112 appears. The lid 113 is screwed to the main body case 112 with four screws 192. An indentation 113a having a semicircular shape is formed in the lid 113. An indentation 112h having a semicircular shape is also formed in a corresponding portion of the main body case 112. For this reason, when the lid 113 is attached to the main body case 112, an opening having a circular shape through which opening the cable 90 goes is formed in that portion. The cable 90 goes through the opening while being surrounded by the rubber ring 92 and enters the second room 11B.

In addition, the pressure sensor 31 is housed in the second room 11B. The pressure sensor 31 includes a cylinder 311. The pressure sensor 31 is a sensor that the pressure sensor 31 is placed in an atmospheric pressure ambiance so as to measure an air pressure inside the cylinder 311. The cylinder 311 is inserted into an opening 112k provided in the main body case 112. The opening 112k is an opening formed in a center of the boss 112f (see FIG. 9) projecting inside the first room 11A. The connector 125 is fitted on the boss 112f. The pressure sensor 31 is provided on the circuit board 30a.

In addition, the flow sensor 32 is also housed in the second room 11B. The flow sensor 32 is a sensor which includes two cylinders 321 and 322 and measures a difference between air pressures inside those two cylinders 321 and 322 to convert to a flow rate of air. These two cylinders 321 and 322 are inserted into two openings 112i and 112j provided in the main body case 112, respectively. These openings 112i and 112j are openings which are provided in centers of the two bosses 112d and 112e (see FIG. 9), respectively. The connectors 123 and 124 are respectively fitted to these bosses 112d and 112e. The flow sensor 32 is provided on the circuit board 30b.

The circuit board 30a on which the pressure sensor 31 is provided is fixed to the circuit board 30b on which the flow sensor 32 is provided, and the relaying board 30 illustrated in FIG. 7 is composed of those two circuit boards 30a and 30b. The air pressure of the inside of the discharging side silencer 60 is transmitted via the silicone tubes 231 to 234 illustrated in FIG. 9 to the cylinder 311 of the pressure sensor 31 and the two cylinders 321 and 322 of the flow sensor 32. Details will be described later.

The cable 90 which connects the blower unit 10 with the control unit 80 illustrated in FIG. 1 includes plural wires 90a, enters the second room 11B and is connected to the relaying board 30. In addition, the wires 91 extending to the circuit board 514 of the turbofan 50 is also connected to the relaying board 30, via a connector 33 provided on the relaying board 30. By means of this, pressures and flow rates measured by the pressure sensor 31 and the flow sensor 32 are transmitted to the control unit 80. In addition, a signal for controlling the rotation of the turbofan 50 which signal comes from a side of the control unit 80 is transmitted, to the circuit board 514 of the turbofan 50 via the relaying board 30, and the turbofan 50 rotates according to the signal.

In addition, two small grooves 113b each having a semicircular shape in addition to the indentation 113a for allowing the cable to go through are formed in the lid 113. In addition, also in the main body case 112, grooves 112m each having a semicircular shape are formed at positions respectively corresponding to the two grooves 113b of the lid 113. When the lid 113 is attached to the main body case 112, the two air openings 11f (see FIG. 5) for holding the second room 11B at atmospheric pressure are formed by those grooves 113b and 112m. The air pressure inside the first room 11A is varied by the operation of the turbofan 50. The second room 11B is configured to be airtight with respect to the first room 11A, and is stably held at atmospheric pressure by the air openings 11f.

The pressure sensor 31 is a sensor that the pressure sensor 31 is placed in an atmospheric pressure ambiance so as to measure an air pressure inside the cylinder 311. In the present embodiment, the second room 11B which is held at atmospheric pressure is provided, and the pressure sensor 31 is arranged inside the second room 11B, and thereby, an air pressure at a targeted place (will be described later) is measured in a high precision manner. Supposing it is intended to measure a pressure in high precision manner without the second room 11B which is held at the atmospheric pressure being provided in the housing 11 as the present embodiment, a configuration in which the pressure sensor 31 is housed in a small airtight box and atmospheric pressure of an outside is guided into an inside of the box with a tube and the like will be required. In the case of the present embodiment, since the second room 11B is provided in the housing 11, there is no requirement for a complicated configuration such as putting a pressure sensor in a box and the like, and there are contributions to size reduction, weight reduction and cost reduction. In addition, in the case of the present embodiment, since electrical components such as the relaying board 30, the pressure sensor 31, the flow sensor 32 and the like are gathered in the second room 11B, it is possible to perform failure inspections of electrics just by opening the lid 113, and thus, maintenance is also improved.

The turbofan 50 applied in the CPAP device 100 according to the present embodiment is a fan 50 including an air dynamic pressure bearing. In other words, a rotor included in the turbofan 50 rotates in a high speed without contacting a stator, and makes a required flow rate of air. In the CPAP device 100 according to the present embodiment, the above-described layout and the application of the turbofan 50 including the air dynamic pressure bearing work together to make it succeed in reducing the size/the weight of the blower unit 10 greatly.

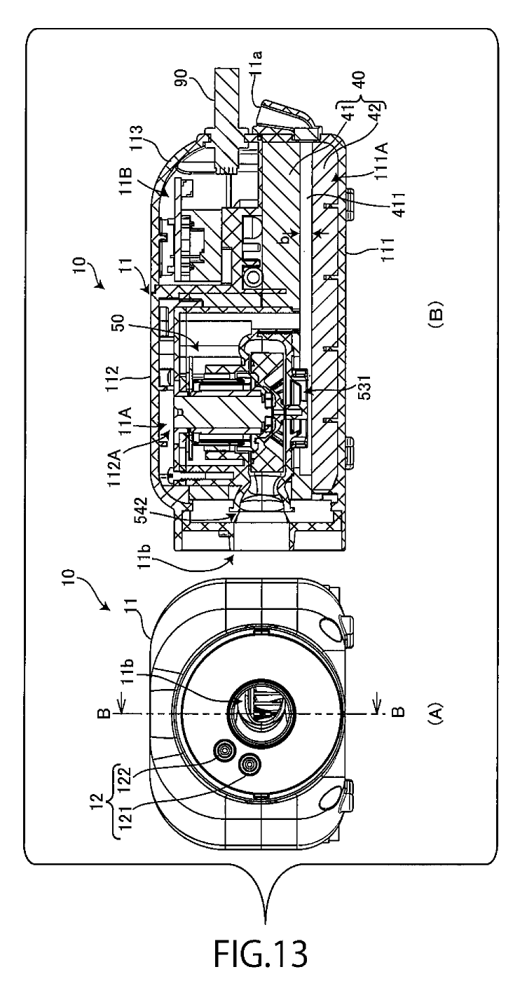

FIG. 13 illustrates a side view of the blower unit when viewed from a side of the air outflow opening (Part (A)), and a cross sectional view along Arrows B-B illustrated in Part (A) of FIG. 13 (Part (B)).

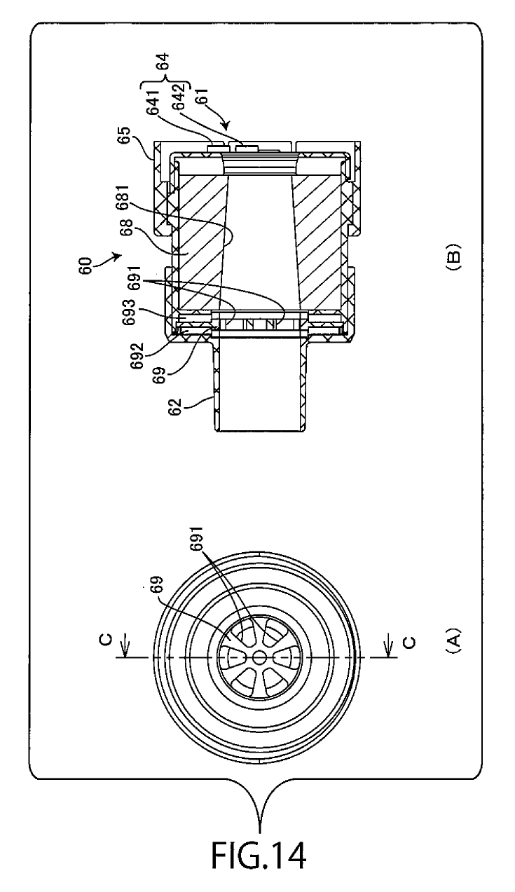

In addition, FIG. 14 illustrates a side view of the discharging side silencer when viewed from a side of the air feeding opening to feed air in the hose (Part (A)), and a cross sectional view along Arrows C-C illustrated in Part (A) of FIG. 14 (Part (B)).

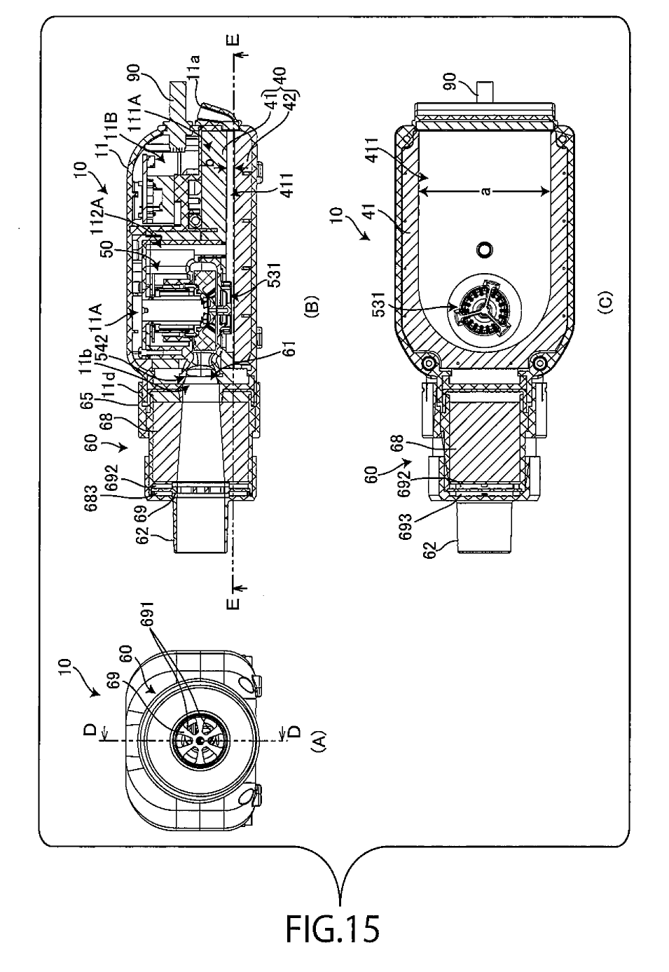

Further, FIG. 15 illustrates a side view of the blower unit in a state of being attached with the silencer and the silencer when viewed from a side of the air feeding opening of the silencer (Part (A)), a cross sectional view along Arrows D-D illustrated in Part (A) of FIG. 15 (Part (B)) and a cross sectional view along Arrows E-E illustrated in Part (B) of FIG. 15 (Part (C)).

As described above, the first room 11A and the second room 11B are provided in the housing 11 of the blower unit 10. The first room 11A includes the first section 111A intersecting a direction of the air flow and vertically overlapping with respect to the second room 11B, and the second section 112A which does not overlap the second room 11B. The drawing side silencer 40 composed of the first absorbing member 41 and the second sound absorbing member 42 is mainly arranged in the first section 111A, and the turbofan 50 is mainly arranged in the second section 112A (see FIG. 9). In addition, the electrical components such as the relaying board 30, the pressure sensor 31 and the flow sensor 32 are arranged in the second room 11B (see FIG. 12).

In addition, the discharging side silencer 60 is connected to the hose 70 (see FIGS. 1 and 2), and is also attached to the blower unit 10 attachably and detachably. The sound absorbing member 68 and the regulating plate 69 are housed in the discharging side silencer 60. An air flow path 681 whose more downstream side in the flow of air is more widened is arranged in the sound absorbing member 68. The sound absorbing member 68 plays a role of receiving air flowing out from the air outflow opening 11b of the blower unit 10 and reducing outflow sounds of the air. In addition, as illustrated in FIG. 14 and Part (A) of FIG. 15, plural openings 691 are provided in the regulating plate 69. The regulating plate 69 plays a role of allowing air to pass through and making the flow of the air after passing through closer to a regulated flow than that before passing through. In the following, the role of the regulating plate 69 will be described in detail.

The air sent out from the blower unit 10 by the turbofan 50 disperses in velocity and direction and is not stable, and thus, vortexes and pressure variations occur in the air flow path. Since the vortexes and pressure variations cause noises and vibrations and further affect breathing easiness of a patient, it is desirable to reduce them to be small. The regulating plate 69 is arranged, and thus, the flow is regulated when the air passes through gaps of the regulating plate 69, and the flow velocity variations and pressure variations are reduced. In addition, vortex occurrences are also blocked by the regulating plate 69, and by means of this, an area where vortexes occur is restricted on an upstream side of the regulating plate 69. Since the regulating plate 69 is arranged and thus the pressure variations, noises therewith and the like are reduced to a smaller amount, it is possible to obtain a required noise reduction rate even if a volume of the sound absorbing member 68 is decreased, and thus, it is possible to reduce the silencer 60 in size/weight while decreasing the volume of the sound absorbing member 68.

However, the regulating plate 69 reduces the flow velocity variations and the pressure variations by producing a pressure loss, and is necessarily accompanied by the pressure loss. Thus, in the present embodiment, turning that to its advantage, the flow rate of the air passing through the regulating plate 69 is measured by measuring a differential pressure before and after the regulating plate 69. A configuration around the regulating plate 69 for the pressure measurement of air will be explained in the following.

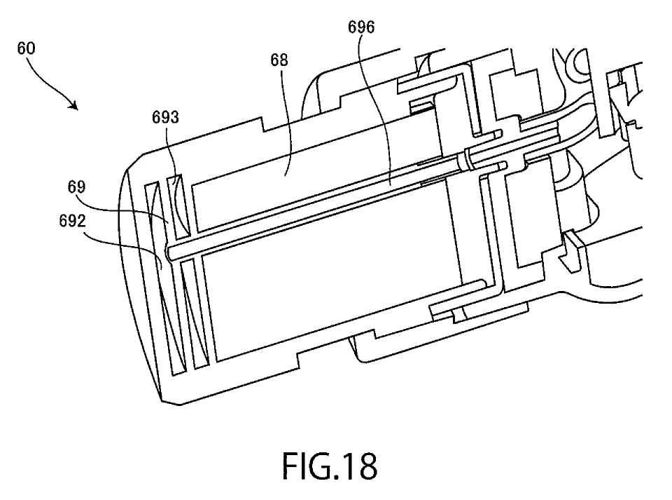

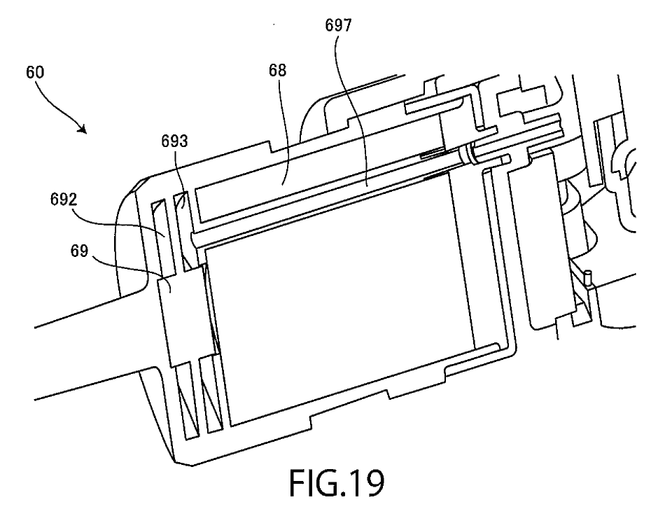

As illustrated in FIGS. 14 and 15, a first air pressure measurement room 692 for the pressure measurement of air which first air pressure measurement room 692 connects to the air flow path immediately after passing through the regulating plate 69, and a second air pressure measurement room 693 which connects to the air flow path immediately before passing through the regulating plate 69 are provided in a circumference of the regulating plate 69. The two connectors 64 (see FIG. 5) which are coupled with the two connectors 12 (see FIG. 4) of the blower unit 10 are provided in the silencer 60. When the two connectors 12 and the two connectors 64 are coupled with each other, respectively, the two air pressure transmitting paths 911 (see FIG. 7) extending between the blower unit 10 and the silencer 60 are formed. One connector 641 (see FIG. 14) of the two connectors 64 provided in the silencer 60 is connected to the second air pressure measurement room 693 by a second air passage 697 (see FIG. 19) extending to an inside of a wall of the silencer 60. Then, the connector 641 is integrated with one connector 121 (see FIGS. 9 and 13) of the two connectors 12 of the blower unit 10. In other words, the air pressure of the second air pressure measurement room 693 is transmitted to the flow sensor 32 (see FIG. 12) via the tube 231 and the connector 123 which are illustrated in FIG. 9. In addition, the other connector 642 (see FIG. 14) of the two connectors 64 provided in the silencer 60 is connected to the first air pressure measurement room 692 by a first air passage 696 (see FIG. 18) extending to the inside of the wall of the silencer 60. And, the connector 642 is integrated with the other connector 122 (see FIG. 9) of the two connectors 12 of the blower unit 10. In other words, the air pressure of the first air pressure measurement room 692 is connected to the tube 232 illustrated in FIGS. 9 and 13, is further connected to the two tubes 233 and 234 by the branching-type connector 126 as illustrate in FIG. 9, and, via each of the connectors 124 and 125, is transmitted to the flow sensor 32 by one of them and to the pressure sensor 31 (see FIG. 12) by the other. By means of this, with the pressure sensor 31, the air pressure of the first air pressure measurement room 692 of the silencer 60, that is, an air pressure of the air after passing through the regulating plate 69 is measured. In addition, with the flow sensor 32, based on the differential pressure between the second air pressure measurement room 693 and the first air pressure measurement room 692 of the silencer 60, that is, the pressure difference of the air immediately before and immediately after passing through the regulating plate 69, the flow rate of the air fed in the hose 70 (see FIGS. 1 and 2) from the silencer 60 is measured.

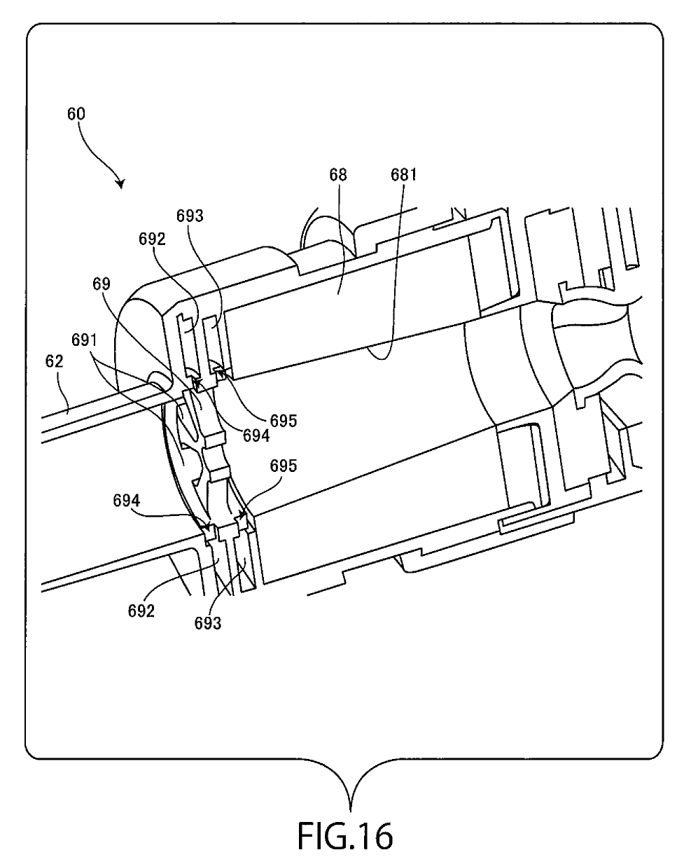

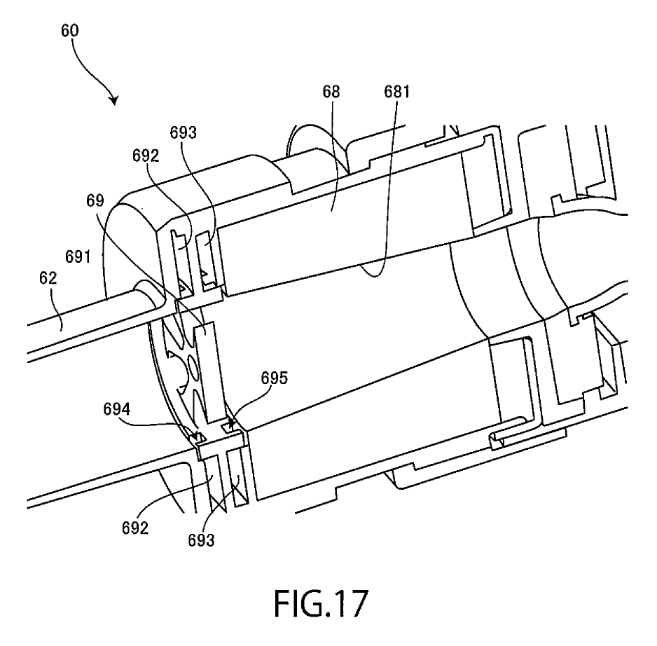

FIGS. 16 and 17 are cross sectional views of a portion of the regulating plate of the silencer.

Here, FIG. 16 and FIG. 17 are slightly different from each other in the cross sectional place.

The first air pressure measurement room 692 and the second air pressure measurement room 593 are partitioned as rooms going around circumferentially to surround the regulating plate 69. And, the first air pressure measurement room 692 is connected to a portion of the air flow path which portion is immediately after passing through the regulating plate 69 by first communicating paths 694 provided at plural places in a circumferential direction. In addition, similarly to this, the second air pressure measurement room 693 is connected to a portion of the air flow path which portion is immediately before passing through the regulating plate 69 by second communicating paths 695 provided at plural places in the circumferential direction. Each of the first communicating paths 694 and the second communicating paths 695 is a substantially small opening, compared with a volume of the first air pressure measurement room 692 or the second air pressure measurement room 693 which are provided at the plural places in the circumferential direction. For this reason, the air pressures of the portions of the air flow path after and before passing through the regulating plate 69 are transmitted to the insides of the first air pressure measurement room 692 and the second air pressure measurement room 693, respectively, and transmission of the air pressure variations of the air flowing through the air flow path is reduced. In other words, an environment in which the respective pressures of the air after and before passing through the regulating plate 69 may be stably measured is formed by the first air pressure measurement room 692 and the first communicating paths 694, and the second air pressure measurement room 693 and the second communicating paths 695.

FIGS. 18 and 19 are cross sectional views of a portion of an end in a radius direction of a portion in the regulating plate of the silencer. FIG. 18 and FIG. 19 are slightly different from each other in the cross sectional position.

In FIGS. 18 and 19, the first air passage 696 and the second air passage 697 each having a tubular shape and respectively extending through the sound absorbing member 68 to the first air pressure measurement room 692 and the second air pressure measurement room 693 are illustrated.

When the silencer 60 is attached to the blower unit 10, the first air passage 696 illustrated in FIG. 18 transmits the air pressure inside the first air pressure measurement room 692 through the tubes 232, 233 and 234 illustrated in FIG. 9 to the flow sensor 32 and the pressure sensor 31 (see FIG. 12). In addition, similarly to this, when the silencer 60 is attached to the blower unit 10, the second air passage 697 illustrated in FIG. 19 transmits the air pressure inside the second air pressure measurement room 693 through the tube 231 illustrated in FIG. 9 to the flow sensor 32 (see FIG. 12). In other words, the first air passage 696 and the second air passage 697 illustrated in FIGS. 18 and 19 carry portions inside the silencer 60 which portions are of the two air pressure transmitting paths 911 (see FIG. 7) extending between the silencer 60 and the blower unit 10.

The air flowing in from the air inflow opening 11a of the blower unit 10 flows in the turbofan 50 from the air intake opening 531 of the turbofan 50 through the air flow path 411 sandwiched by the two sound absorbing members 41 and 42. The air which has flown in the turbofan 50 is discharged from the air discharging opening 542 of the turbofan 50 by the rotation of the turbofan 50, flows out from the air outflow opening 11b of the blower unit 10, flows in the discharging side silencer 60, and is further fed in the mask 200 (see FIG. 2) via the hose 70.

Incidentally, as the flow sensor 32, one in which the flow rate is converted from the pressure difference between the first air pressure measurement room 692 and the second air pressure measurement room 693 has been explained, and however, one in which a pressure is measured by a method other than that may be used, and for example, a thermal flow sensor using a heater may be used.

Next, the drawing side silencer 40 (see FIG. 7, Part (B) of FIG. 13, Part (B) and Part (C) of FIG. 15) housed in the blower unit 10 will be studied.

The drawing side silencer 40 is composed of the two sound absorbing members 41 and 42 which are arranged vertically while sandwiching the air flow path 411 having the tabular shape. As described above, the air flow path 411 sandwiched by the two sound absorbing members 41 and 42 has the tabular shape of the width a (see FIG. 9 and Part (C) of FIG. 15) by the height b (see FIG. 9, Part (B) of FIG. 13 and Part (B) of FIG. 15).

Here, regarding a silencer of a configuration in which a flow path of air surrounded by a sound absorbing member is formed, desirable shapes will be discussed in the following view point.

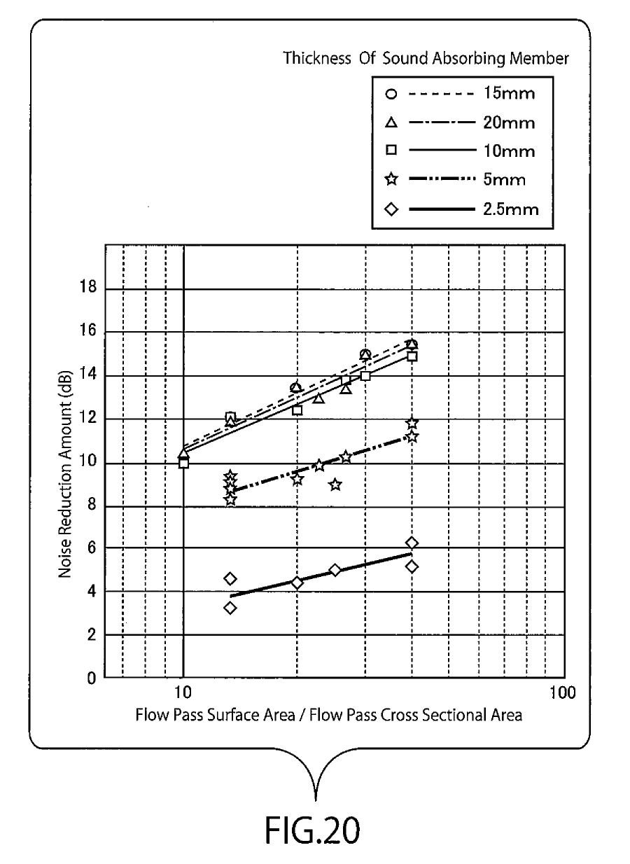

FIG. 20 is a view illustrating a sound absorbing performance when a length of the flow path, a cross sectional shape and a thickness of the sound absorbing member are changed in various kinds by the inventors of the present invention.

Based on results of this experiment, it is obtained that the sound absorbing performance is represented by the equation (1) with a sound absorbing coefficient Cm which is determined by material and the thickness of the sound absorbing member, a flow path cross sectional area Sa and a flow path surface area Ss.

ΔN=Cm·(Ss/Sa) (1)

In the following, desirable flow path cross sectional shapes will be studied by using this relationship.

When it is assumed that the cross sectional shape is a rectangle of a width a by a height b, and a length of the flow path is 1,

Sa=a·b (2)

Ss=2·(a+b)·l (3)

are obtained. Using a parameter t representing the cross sectional shape (a cross sectional shape coefficient), a and b are expressed as:

a=\sqrt{Sa}·t (4)

b=\sqrt{Sa}/t (5)

The shape will be a square when t=1, the larger t is the larger the width is when t>1, the smaller t is the larger the height is when t<1, and the area becomes constantly Sa regardless of t.

When the sound absorbing performance ΔN is expressed using the equations (1) to (5),

ΔN=2·(Cm/\sqrt{Sa})·l·(t+1/t) (6)

is obtained.

Incidentally, regarding the thickness of the sound absorbing member, in the case of the sound absorbing member used here, 5 mm or more is desirable, and 10 mm is a sufficient thickness requiring no further being thickened.

(Regarding Flow Path Resistance)

Next, from a view point of flow path resistance, desirable cross sectional shapes will be studied.

When it is assumed that a tube friction coefficient is A, a tube length is l, a diameter is d, a density is ρ and a flow velocity is u, a pressure loss ΔP by a flow path resistance of a circular tube at the time of a laminar flow becomes

ΔP=λ·(l/d)·ρ·(u²/2) (7)

In addition, an equivalent circular tube diameter of a rectangular cross sectional flow path de becomes

de=1.3·((a·b)⁵/(a+b)²)^{0.125} (8)

From the equations (7), (8), (4) and (5),

ΔP=(1/2.6)·λ·ρ·(u²/Sa^{0.5})·l·(t+l/l)^{0.25} (9)

is obtained.

(Regarding Volume)

From the equations (6) and (9), the longer the flow path length l is the larger each of the sound absorbing performance and the resistance is, and the smaller the cross sectional area Sa is the larger each of the sound absorbing performance and the resistance is.

Here, optimizing the cross sectional shape will be considered. For this, the consideration will be performed while things other than the cross sectional shape coefficient t of the equations (6) and (9) are fixed. If a cross sectional shape in which the flow path resistance is small and the sound absorbing performance is large is found, it is possible by using that shape to select a flow path length 1 and a cross sectional area Sa which make the volume be as small as possible, in a range of allowable flow path resistance and allowable noise.

(Discussion of Cross Sectional Shape Parameter t)

When it is assumed that the sound absorbing performance ΔN and the flow path loss ΔP when the cross sectional shape is square, that is, when t=1, are ΔN₁ and ΔP₁,

ΔN/ΔN₁=(t+1/t)₂ (10)

ΔP/ΔP₁=(t+1/t)^{0.25}/2 (11)

are obtained.

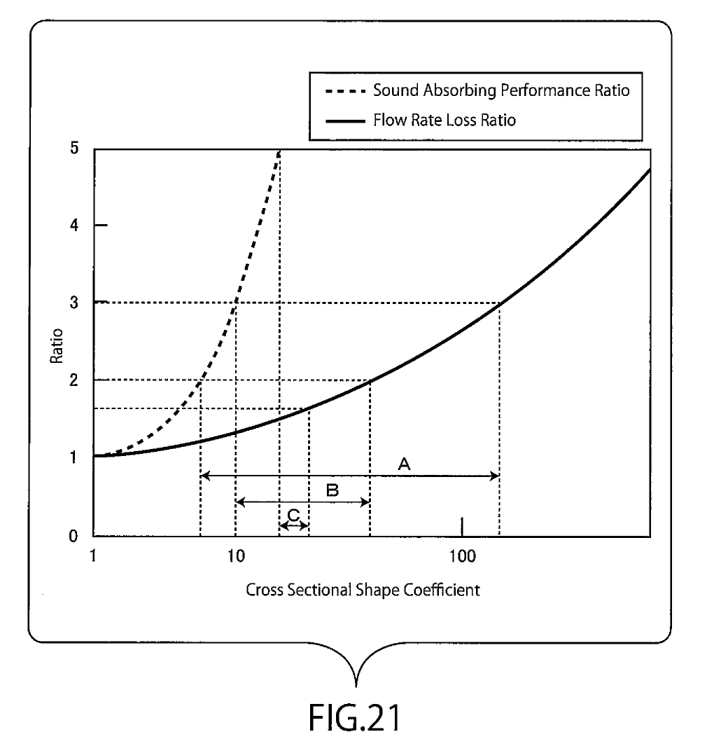

FIG. 21 is a view illustrating a sound absorbing performance ratio and a flow rate loss ratio with respect to the cross sectional shape coefficient t. In this FIG. 21, the horizontal axis represents the cross sectional shape coefficient t plotted in a logarithmic scale. Since the graph becomes symmetrical on the right side and the left side across t=1, this FIG. 17 illustrates only an area of t≧1.

Here, a range of an appropriate cross sectional shape will be considered as follows.

If the ratio ΔN/ΔN₁ of the sound absorbing performance to that of the square is:
A. fivefold or more, since this corresponds to a noise reduction of 7 dB of more, it is recognized that an effect of the shape is exhibited quite well, and in this case, approximately t≧10;
B. threefold or more, since this corresponds to noise reduction of 5 dB or more, it is recognized that an effect of the shape is exhibited well, and in this case, approximately t≧6; and
C. twofold or more, this corresponds to noise reduction of 3 dB or more, and an effect of the shape is recognized, and in this case, approximately t≧4.

If the ratio ΔP/ΔP₁ of the flow path loss to that of the square is:
A. 1.7 or less, this may be used without problems. In this case, approximately
B. 2 or less, this may be used depending on designing condition of the flow path. In this case, approximately t≦30.
C. 3 or less, this may be used through carefully considering designing conditions of the flow path. In this case, approximately t≦160.

The flow path designing condition referred to in here represents characteristics of the fan and a shape of the flow path from a drawing-in opening to the mask via a hose, for satisfying (a producible pressure of the turbofan at the time of a maximum use flow rate−a pressure loss produced in the flow path at the maximum use flow rate)>a pressure required for usage.

The foregoing is integrated as follows: desirably, 4≦t≦160 (a range A illustrated in FIG. 27); further desirably, 6≦t≦30 (a range B illustrated in FIG. 27); and furthermore desirably, 10≦t≦16 (a range C illustrated in FIG. 27).

This ends the explanation of the basic one embodiment according to the present invention, and various modified examples will be explained in the following. Also in the following, elements common to those in the above-described embodiment are given same reference signs as those in the embodiment even if there are differences in the shape and the like, and explanations thereof are omitted.

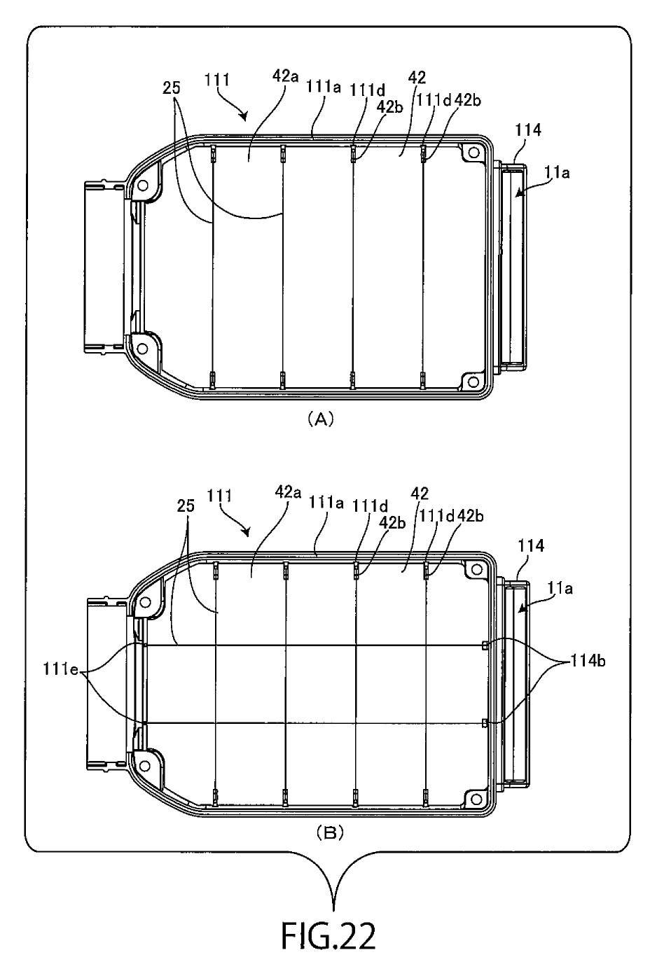

FIG. 22 is a view illustrating modified examples of wire stretching way to reduce deformations of the second sound absorbing member. FIG. 22 is a view corresponding to FIG. 11 in the above-described embodiment.

Two examples in which the stretching way of the wire 25 to reduce deformations of the second sound absorbing member 42 is changed are illustrated in here. The wire 25 just has to reduce deformations of the second sound absorbing member 42, and may be stretched around as illustrated in FIG. 11 or may be stretched around as illustrated in Part (A) of FIG. 22 or Part (B) of FIG. 22.

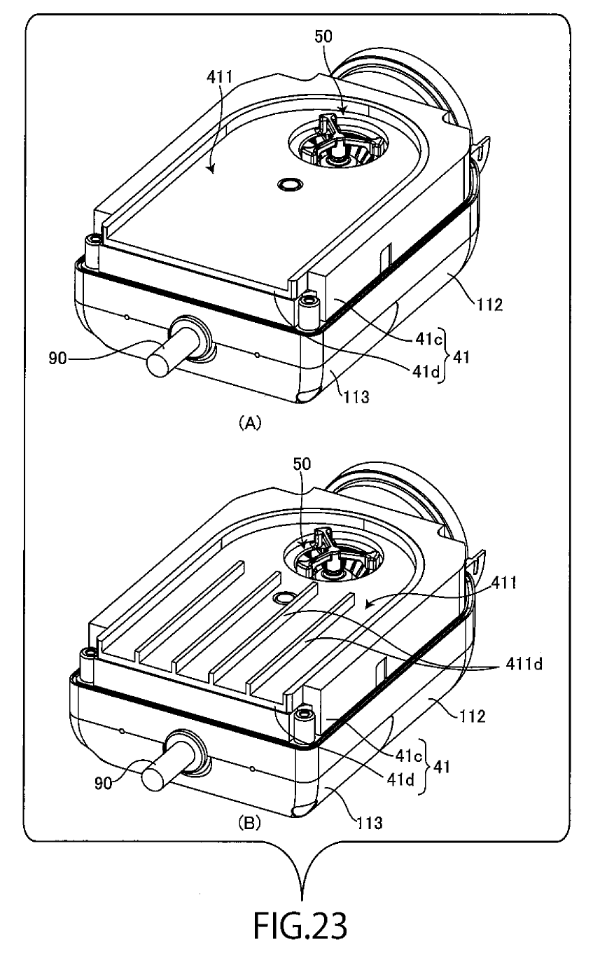

FIG. 23 is a view illustrating modified examples of the first sound absorbing member.

In the above-described embodiment, as the first sound absorbing member 41, the sound absorbing member made of single material and having the shape illustrated in FIG. 9 is applied. Air flows in the air flow path 411, so that a force in a direction to close the air flow path 411 is applied to the first sound absorbing member 41. In the above-described embodiment, the sound absorbing member made of material having hardness enough to counter the force and avoid the deformations is applied. In contrast, a first sound absorbing member 41 in Part (A) of FIG. 23 is composed of a base 41c made of sound absorbing material having soft quality and a surface forming layer 41d made of sound absorbing material having the relatively harder quality to sustain a force to close an air flow path 411 which surface forming layer is overlapped on the base 41c. The surface forming layer 41d forms a lower surface of upper and lower surfaces which form the air flow path 411 and are separated from each other by a distance b. As described, only the surface forming layer 41d which forms the air flow path 411 is configured with the the sound absorbing material having the relatively harder quality and the base 41c is configured with the sound absorbing material having the soft quality, so that it is possible to improve the sound absorbing performance of a drawing side silencer 40 configured with this first sound absorbing member 41 and the second absorbing member 42 illustrated in FIG. 10.

In a first sound absorbing member 41 illustrated in Part (B) of FIG. 23, in addition to the two-layer configuration of Part (A) of FIG. 23, ribs 411d projecting toward an upper surface (the surface 42a of the second sound absorbing member 42 illustrated in FIG. 10) facing the surface forming layer 41d are further provided. The ribs 411d are provided, and thus, even though the first sound absorbing member 41 begins to deform, the ribs 411d hit against the second sound absorbing member 42 (see FIG. 10) so that deformations are reduced, and an air flow path 411 is secured further securely, compared with Part (A) of FIG. 23.

Incidentally, in this Part (B) of FIG. 23, the example in which the ribs 411d are provided is illustrated, and however, bosses or projections having a post shape instead of the ribs may be applied, and the shape of the projections is not limited.

In addition, this Part (B) of FIG. 23 represents the example in which the ribs 411d are provided in the first sound absorbing member 41 having the two-layer configuration of the base 41c and the surface forming layer 41d, and however, the two-layer configuration is necessarily required in order to provide projections such as the ribs 411d and the like, and a first sound absorbing member in which projections are provided may be formed by using a sound absorbing material made of single kind of material.

Further, the example in which the two-layer configuration or the projection configuration is applied to the first sound absorbing member 41 are illustrated in here, and however, these configurations may be applied to the second sound absorbing member 42 (see FIG. 10). In that case, they may be used together with the reduction of deformation by the wire 25 illustrated in FIG. 11, or a configuration without the wire 25 may be used.

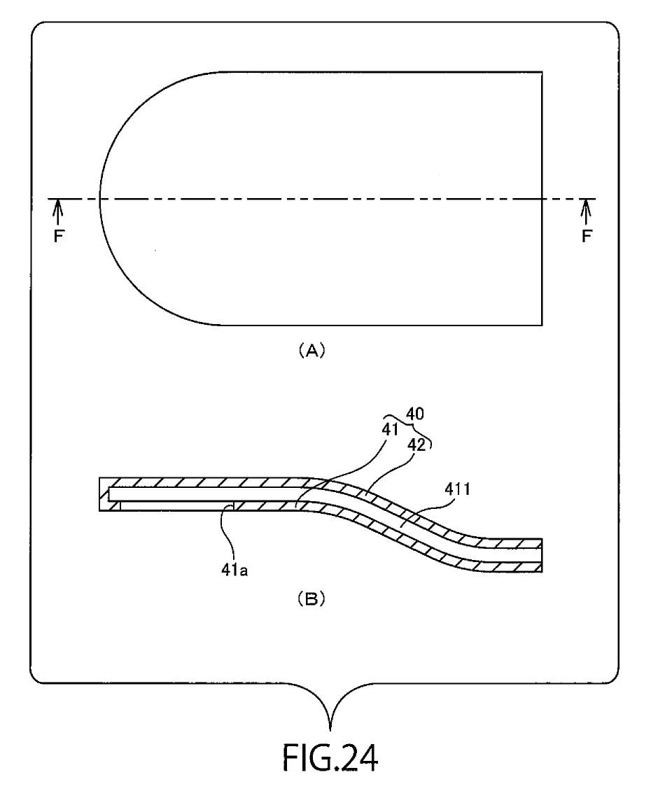

FIG. 24 is a view illustrating a modified example of a drawing side silencer.

Here, Part (A) FIG. 24 is a plan view, Part (B) of FIG. 24 is a cross sectional view along Arrows F-F illustrated in Part (A) of FIG. 24.

The drawing side silencer 40 in the above-described embodiment is a silencer in which the air flow path 411 having the tabular shape is formed. In contrast, an air flow path 411 of a drawing side silencer 40 illustrated in this FIG. 24 has a shape in which a flat plate is gently bent. It is desirable that the drawing side silencer 40 has an air flow path having the tabular shape, and however, depending on a layout of components and the like, the drawing side silencer 40 may have the air flow path 411 having a gently curved plate shape as illustrated in this FIG. 20.

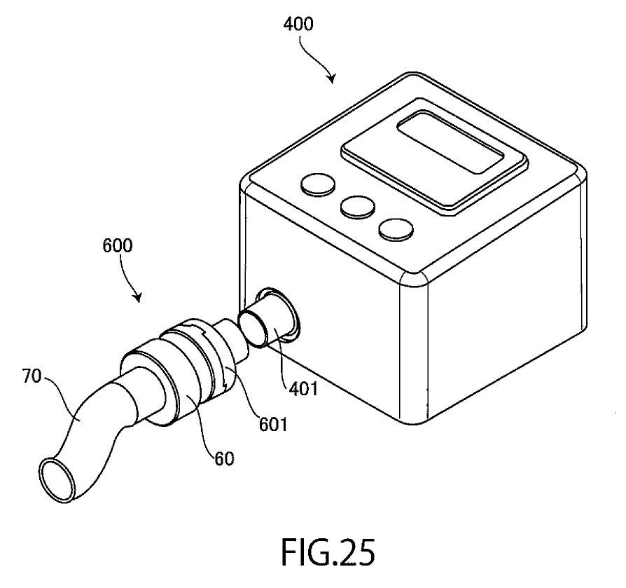

FIG. 25 is a perspective view illustrating a modified example of the discharging side silencer.

A CPAP device 400 different from one in the above-described embodiment is illustrated in here. The CPAP device 400 is irrelevant to whether or not a feature according to the present invention is included, and for example, may be a CPAP device of conventional type. Also in the CPAP device 400, there exists an air discharging opening 401 which has a shape projecting cylindrically and is to be connected to a hose 70. The standard is established for the hose 70, and the air discharging opening 401 has a shape which is to be fitted into the hose 70 having a size conforming to the standard.

A discharging side silencer 600 illustrated in here is one in which an adapter 601 which is coupled to each of the air discharging opening 401 and the silencer 60 in the above-described embodiment is attached to the silencer 60. Such adapter 601 is attached to the silencer 60 in the above-described embodiment, so that the silencer 600 is interposed between the CPAP device 400 to which the hose 70 is to be directly connected and the hose 70, and thus, it is possible to reduce outflow noise of air.

Incidentally, the adapter 601 is attached to the silencer 60 of the above-described embodiment to make the new silencer 600 in here, and however, a silencer may be configured as a type in which a sound absorbing structure is provided inside, which is connected to each of the hose 70 and the air discharging opening 401 to which the hose 70 of the CPAP device 400 is to be connected, and which, at the time of normal storing, is separated from the CPAP device 400 and is remained being attached to the hose 70.

In addition, the discharging side silencer 60 in the above-described embodiment is a silencer in which the sound absorbing member 68 (see FIGS. 14 and 15) is housed so as to obtain the sound absorbing effect, and however, the discharging side silencer 60 may be a silencer having a washable chamber configuration. In that case, it is also possible to wash the silencer together with the hose 70 while being connected to the hose 70.

As explained, instead of the above-described embodiment, various modified examples may be applied.

### REFERENCE SIGNS LIST

10 Blower Unit
11 Housing
11A First Room
11B Second Room
11a Air Inflow Opening
11b Air Outflow Opening
11c Attaching Surface
11d Coupling Cylinder
11e Locking Projection
12, 33 Connector
20 Air Filter
21 Grommet
22 Round Cross Section String
25 Wire
30 Relaying Board
31 Pressure Sensor
32 Flow Sensor
40 Drawing Side Silencer
41, 42, 68 Sound Absorbing Member
42a Surface
41a, 41b Opening
41c Base
41d Surface Forming Layer
50 Turbofan
60, 600 Silencer
61 Air Receiving Opening
62 Air Feeding Opening
63 Attaching Surface
64 Connector
65 Coupling Cylinder
66 Locking Opening
67 Nick
69 Regulating Plate
70 Hose
80 Control Unit
81 User Interface
81a Operation Button
81b Display Screen
82 Control Board
83 Battery
84 AC adaptor Connecting Terminal
90 Cable
90a, 91 Wire
92 Rubber Ring
100, 400 CPAP Device
111 Bottom Case
111b Air Intake Opening
111c, 411d Rib
112 Main Body Case
112a Bottom Wall
112b Standing Wall
112m, 112c, 113b Groove
112d, 112e, 112f Boss
112h, 113a Indentation
112i, 112j Opening
113 Lid
114 Drawing Opening Cover
115 Discharging Opening Cover
64, 122, 123, 124, 125, 126, 641 Connector
191, 192 Screw
200 Mask
231, 232, 233, 234 Silicone Tube
311, 321, 322 Cylinder
401 Air Discharging Opening
411, 681 Air Flow Path
514 Circuit Board
515 Connector
591 Projection
601 Adapter
691 Opening
692 First Air Pressure Measurement Room
693 Second Air Pressure Measurement Room
694 First Communicating Path
695 Second Communicating Path
696 First Air Passage
697 Second Air Passage
911 Air Pressure Transmitting Path

1. A CPAP device comprising:
a fan that draws in air and sends out the air; and
a silencer that reduces an outflow sound of the air sent out from the fan, and feeds the air whose outflow sound is reduced in a hose connected to a mask or a nasal cannula attached to a patient, wherein
the silencer includes a regulating plate which allows the air to pass therethrough so as to make a flow of the air after the passing closer to a regulated flow than that before the passing.

2. The CPAP device according to claim 1, further comprising:
a pressure sensor that measures a pressure of the air after passing through the regulating plate; and
a control circuit that controls a rotation speed of the fan according to the pressure of the air measured by the pressure sensor.

3. The CPAP device according to claim 2, comprising:
a first air pressure measurement room that is to be a measurement target for the pressure of the air by the pressure sensor; and
a first communication path that connects the air flow path after passing through the regulating plate with the first air pressure measurement room.

4. The CPAP device according to claim 2, comprising:
a flow sensor that measures a flow rate of the air flowing while passing through the regulating plate based on a differential pressure of the air before and after passing through the regulating plate, wherein
the control circuit controls the rotation speed of the fan according to both the pressure of the air measured by the pressure sensor and the flow rate of the air measured by the flow sensor.

5. The CPAP device according to claim 4, comprising:
a second air pressure measurement room for measuring the differential pressure of the air by the flow sensor; and
a second communicating path that connects the air flow path before passing through the regulating plate with the second air pressure measurement room, wherein
the flow sensor measures the flow rate of the air flowing while passing through the regulating plate based on a differential pressure between the air inside the second air pressure measurement room and the air after passing through the regulating plate.

6. The CPAP device according to claim 1, comprising:
a housing which houses the fan and in which an air outflow opening to allow the air sent out from the fan to flow out is formed, wherein
the silencer is a silencer which is provided separately from the housing, is attached to one end of the hose and is attachably and detachably attached to the air outflow opening.

7. The CPAP device according to claim 2, comprising:
a housing which houses the fan and the pressure sensor and in which an air outflow opening to allow the air sent out from the fan to flow out is formed, wherein
the silencer is a silencer which is provided separately from the housing, is attached to one end of the hose and is attachably and detachably attached to the air outflow opening, and
the silencer includes a first air pressure transmitting path which is coupled to extend between a side of the silencer and a side of the housing by the attachment of the silencer to the air outflow opening, and transmits the pressure of the air after passing through the regulating plate to the pressure sensor.

8. The CPAP device according to claim 4, comprising:
a housing which houses the fan, the pressure sensor and the flow sensor, and in which an air outflow opening to allow the air sent out from the fan to flow out is formed, wherein,
the silencer is a silencer which is provided separately from the housing, is attached to one end of the hose and is attachably and detachably attached to the air outflow opening, and
the silencer includes:
a first air pressure transmitting path which is coupled to extend between the silencer and the housing by the attachment of the silencer to the air outflow opening, and transmits the pressure of the air after passing through the regulating plate to the pressure sensor and the flow sensor; and
a second air pressure transmitting path which is coupled to extend between a side of the silencer and the housing by the attachment of the silencer to the air outflow opening, and transmits a pressure of the air before passing through the regulated plate to the flow sensor.
